(19) United States
(12) Patent Application Publication
(10) Pub. No.: US 2023/0285469 A1
(43) Pub. Date: Sep. 14, 2023

(54) Therapeutic Use of Cell-free Fat Extract Solution for Pulmonary Diseases

(71) Applicant: SHANGHAI SEME CELL TECHNOLOGY CO., LTD., Shanghai (CN)

(72) Inventors: Wenjie ZHANG, Shanghai (CN); Xifan HOU, Shanghai (CN)

(21) Appl. No.: 18/040,466

(22) PCT Filed: Aug. 2, 2021
(86) PCT No.: PCT/CN2021/110119

§ 371 Date: Feb. 3, 2023

(30) Foreign Application Priority Data
Aug. 3, 2020 (CN) 202010767957.4

(51) Int. Cl. A61K 35/35; A61P 11/00

(52) U.S. Cl. CPC A61K 35/35; A61P 11/00

(57) ABSTRACT

The present invention relates to the therapeutic use of a cell-free fat extract solution for pulmonary diseases. In particular, provided in the present invention is the use of a cell-free fat extract in the preparation of a composition or a preparation. The composition for preparation is used for preventing and/or treating pulmonary fibrosis. The cell-free fat extract solution of the present invention has an excellent therapeutic effect on pulmonary fibrosis.

### TECHNICAL FIELD

The invention relates to the field of medicine, in particular to the therapeutic use of cell-free fat extract solution for pulmonary diseases.

### BACKGROUND

Pulmonary fibrosis (pulmonary fibrosis, PF) is a chronic progressive fibrosis caused by smoking, viral infection, environmental pollution, genetic susceptibility and drugs. It is the most common pulmonary interstitial diseases and one of the most serious respiratory diseases.

Its pathological features are mainly diffuse inflammatory cell infiltration, fibroblast proliferation, abnormal deposition of extracellular matrix, and serious destruction of alveolar structure. The condition of PF patients is often progressive, and most of them die in advanced stages due to cardiopulmonary failure. At present, the pathogenesis of PF is still unclear, and there is a lack of effective therapeutic drugs in clinical practice.

Therefore, there is a need in the field to develop a drug that can effectively treat pulmonary fibrosis.

### SUMMARY OF THE INVENTION

The purpose of the present invention is to provide a use of a cell-free fat extract in the prevention and/or treatment of pulmonary fibrosis.

In a first aspect, the present invention provides a use of a cell-free fat extract for the preparation of a composition or formulation for the prevention and/treatment of pulmonary fibrosis.

In another preferred embodiment, the pulmonary fibrosis comprises pulmonary fibrosis caused by cytotoxic antibiotics.

In another preferred embodiment, the pulmonary fibrosis comprises pulmonary fibrosis caused by anti-tumor drugs.

In another preferred embodiment, the anti-tumor drug comprises a chemotherapeutic drug.

In another preferred embodiment, the anti-tumor drug comprises a cytotoxic drug.

In another preferred embodiment, the anti-tumor drug comprises a cytotoxic antibiotic.

In another preferred embodiment, the cytotoxic antibiotic comprises bleomycin.

In another preferred embodiment, the anti-tumor drug comprises bleomycin.

In another preferred embodiment, the pulmonary fibrosis is bleomycin-induced or -caused pulmonary fibrosis.

In another preferred embodiment, the prevention and/or treatment of pulmonary fibrosis comprises prevention and/or treatment in one or more ways selected from the group consisting of:
(i) reducing lung weight index;
(ii) improving peribronchial, bronchio-interstitial and/or pulmonary alveolar inflammatory cell infiltration of a lung;
(iii) improving peribronchial and/or bronchio-interstitial fibrosis of a lung; and/or
(iv) improving peribronchial edema of a lung.

In another preferred embodiment, the cell-free fat extract is a cell-free fat extract obtained from fat in human or non-human mammals.

In another preferred embodiment, the non-human mammal is a monkey, an orangutan, a cow, a pig, a dog, a sheep, a rat or a rabbit.

In another preferred embodiment, the composition or preparation comprises a pharmaceutical composition or preparation, a food composition or preparation, a health care composition or preparation, or a dietary supplement.

In another preferred embodiment, the composition or preparation further comprises a pharmaceutically, food, health care product or a dietary acceptable carrier.

In another preferred embodiment, the dosage form of the composition or preparation is an oral preparation, a topical preparation or an injection preparation.

In another preferred embodiment, the injection preparation is an intravenous injection preparation.

In another preferred embodiment, the composition or preparation is administered by topical, local, or subcutaneous injection.

In another preferred embodiment, the cell-free fat extract is free of cells and free of lipid droplets.

In another preferred embodiment, the lipid droplets are oil droplets released after the fat cells are broken.

In another preferred embodiment, the “free of lipid droplets” means that the volume of oil droplets in the cell-free fat extract is less than 1%, preferably less than 0.5%, more preferably less than 0.1% in total liquid percentage.

In another preferred embodiment, the cells are selected from the group consisting of endothelial cells, adipose stem cells, macrophage cells, and stromal cells.

In another preferred embodiment, the “cell-free” means that the average number of cells in 1 ml of cell-free fat extract is ≤1, preferably ≤0.5, more preferably ≤0.1, or 0.

In another preferred embodiment, the cell-free fat extract is a naturally obtained nano-fat extract with additive-free.

In another preferred embodiment, the “additive-free” means that no solution, solvent, small molecule, chemical agent, and biological additive are added during the preparation of the fat extract except for the rinsing step.

In another preferred embodiment, the cell-free fat extract is obtained by centrifuging the fat tissue after emulsification.

In another preferred embodiment, the fat extract contains one or more components selected from the group consisting of: IGF-1, BDNF, GDNF, TGF-β, HGF, bFGF, VEGF, TGF-β1, HGF, PDGF, EGF, NT-3, GH, G-CSF, and combinations thereof.

In another preferred embodiment, the cell-free fat extract contains, but is not limited to, one or more components selected from the group consisting of IGF-1, BDNF, GDNF, bFGF, VEGF, TGF-β1, HGF, PDGF, and combinations thereof.

In another preferred embodiment, the cell-free fat extract is a cell-free fat extract liquid.

In another preferred embodiment, in the cell-free fat extract, the concentration of the IGF-1 is 5000-30000 pg/ml, preferably 6000-20000 pg/ml, more preferably 7000-15000 pg/ml, more preferably 8000-12000 pg/ml, more preferably 9000-11000 pg/ml, more preferably 9500-10500 pg/ml.

In another preferred embodiment, in the cell-free fat extract, the concentration of BDNF is 800-5000 pg/ml, preferably 1000-4000 pg/ml, more preferably 1200-2500 pg/ml, more preferably 1400-2000 pg/ml, more preferably 1600-2000 pg/ml, more preferably 1700-1850 pg/ml.

In another preferred embodiment, in the cell-free fat extract, the concentration of GDNF is 800-5000 pg/ml, preferably 1000-4000 pg/ml, more preferably 1200-2500 pg/ml, more preferably 1400-2000 pg/ml, more preferably 1600-2000 pg/ml, more preferably 1700-1900 pg/ml.

In another preferred embodiment, in the cell-free fat extract, the concentration of bFGF is 50-600 pg/ml, preferably 100-500 pg/ml, more preferably 120-400 pg/ml, more preferably 150-300 pg/ml, more preferably 200-280 pg/ml, more preferably 220-260 pg/ml.

In another preferred embodiment, in the cell-free fat extract, the concentration of VEGF is 50-500 pg/ml, preferably 100-400 pg/ml, more preferably 120-300 pg/ml, more preferably 150-250 pg/ml, more preferably 170-230 pg/ml, more preferably 190-210 pg/ml.

In another preferred embodiment, in the cell-free fat extract, the concentration of TGF-β1 is 200-3000 pg/ml, preferably 400-2000 pg/ml, more preferably 600-1500 pg/ml, more preferably 800-1200 pg/ml, more preferably 800-1100 pg/ml, more preferably 900-1000 pg/ml.

In another preferred embodiment, in the cell-free fat extract, the concentration of HGF is 200-3000 pg/ml, preferably 400-2000 pg/ml, more preferably 600-1500 pg/ml, more preferably 600-1200 pg/ml, more preferably 800-1000 pg/ml, more preferably 850-950 pg/ml.

In another preferred embodiment, in the cell-free fat extract, the concentration of PDGF is 50-600 pg/ml, preferably 80-400 pg/ml, more preferably 100-300 pg/ml, more preferably 140-220 pg/ml, more preferably 160-200 pg/ml, more preferably 170-190 pg/ml.

In another preferred embodiment, the weight ratio of the IGF-1 to VEGF is 20-100:1, preferably 30-70:1, more preferably 40-60:1, and most preferably 45-55:1.

In another preferred embodiment, the weight ratio of BDNF to VEGF is 2-20:1, preferably 4-15:1, more preferably 6-12:1, and most preferably 8-9.5:1.

In another preferred embodiment, the weight ratio of GDNF to VEGF is 2-20:1, preferably 4-15:1, more preferably 6-12:1, and most preferably 8.5-9.5:1.

In another preferred embodiment, the weight ratio of bFGF to VEGF is 0.2-8:1, preferably 0.5-5:1, more preferably 0.6-2:1, more preferably 0.8-1.6:1, and most preferably 1-1.5:1.

In another preferred embodiment, the weight ratio of TGF-β1 to VEGF is 1-20:1, preferably 1-15:1, more preferably 1-10:1, more preferably 2-8:1, more preferably 4-6:1.

In another preferred embodiment, the weight ratio of HGF to VEGF is 1-20:1, preferably 1-15:1, more preferably 1-10:1, more preferably 2-8:1, more preferably 4-5.5:1.

In another preferred embodiment, the weight ratio of PDGF to VEGF is 0.1-3:1, preferably 0.2-2:1, more preferably 0.4-1.5:1, and most preferably 0.7-1.2:1.

In another preferred embodiment, the cell-free fat extract is prepared by the following method:

(1) providing an fatty tissue raw material, cutting the fatty tissue raw material and rinsing it (e.g., with normal saline) to obtain a rinsed fatty tissue;

(2) centrifuging the rinsed fatty tissue to obtain a layered mixture;

(3) for the layered mixture, the upper oil layer and the lower water layer are removed, and collecting the intermediate layer (i.e. the fat layer containing fat cells);

(4) emulsifying the intermediate layer to obtain an emulsified fat mixture (also called nano-fat);

(5) centrifuging the emulsified fat mixture, thereby obtaining an intermediate liquid layer, i.e. a primary fat extract; and

(6) filtering and de-bacterizing the primary fat extract, thereby obtaining the cell-free fat extract.

The second aspect of the present invention provides a method for preparing a cell-free fat extract, and the method comprises the steps of:

(1) providing an fatty tissue raw material, crushing the fatty tissue raw material and rinsing it (e.g., with normal saline) to obtain a rinsed fatty tissue;

(2) centrifuging the rinsed fatty tissue to obtain a layered mixture;

(3) for the layered mixture, the upper oil layer and the lower water layer are removed, and collecting the intermediate layer (i.e. the fat layer containing fat cells);

(4) emulsifying the intermediate layer to obtain an emulsified fat mixture (also called nano-fat);

(5) centrifuging the emulsified fat mixture, thereby obtaining an intermediate liquid layer, i.e. a primary fat extract; and

(6) filtering and de-bacterizing the primary fat extract, thereby obtaining the cell-free fat extract.

In another preferred embodiment, the cell-free fat extract is as described in the first aspect of the present invention.

In another preferred embodiment, in the step (2), the centrifugation is performed at 800-2500 g, preferably 800-2000 g, more preferably 1000-1500 g, and most preferably 1100-1300 g.

In another preferred embodiment, in the step (2), the centrifugation time is 1-15 min, preferably 1-10 min, more preferably 1-8 min, and most preferably 1-5 min.

In another preferred embodiment, the temperature of the centrifugation is 2-6° C.

In another preferred embodiment, in the step (4), the emulsification is mechanical emulsification.

In another preferred embodiment, the mechanical emulsification is performed by repeated blowing by a syringe (e.g., blowing 20-200 times, preferably 20-150 times, more preferably 20-100 times, more preferably 30-50 times).

In another preferred embodiment, the blowing method is that two 10 ml injection syringes are connected to a tee tube and repeatedly push at a constant speed.

In another preferred embodiment, in the step (4), the emulsification is by means of crushing through a tissue homogenizer.

In another preferred embodiment, the step (5) further includes freezing and thawing the emulsified fat mixture before the centrifugation treatment.

In another preferred embodiment, the thawed mixture is used for centrifugation after freezing and thawing treatment.

In another preferred embodiment, the freezing temperature is from −50° C. to −120° C., preferably from −60° C. to −100° C., more preferably from −70° C. to −90° C.

In another preferred embodiment, the thawing temperature is 20-40° C., preferably 25-40° C., more preferably 37° C.

In another preferred embodiment, the number of cycles of thawing after freezing is 1-5 (preferably 1, 2, 3 or 4).

In another preferred embodiment, in the step (5), after centrifugation, the emulsified fat mixture is layered into four layers, the first layer is an oil layer, the second layer is a residual fatty tissue layer, the third layer is a liquid layer (i.e., an intermediate liquid layer), and the fourth layer is a cell/tissue debris precipitation layer.

In another preferred embodiment, in the step (5), the centrifugation is performed at 800-2500 g, preferably 800-2000 g, more preferably 1000-1500 g, and most preferably 1100-1300 g.

In another preferred embodiment, in the step (5), the centrifugation time is 1-15 min, preferably 1-10 min, more preferably 2-8 min, and most preferably 3-7 min.

In another preferred embodiment, in the step (5), the first layer, the second layer, the third layer and the fourth layer are sequentially arranged from top to bottom.

In another preferred embodiment, in the step (5), the intermediate liquid layer is a transparent or substantially transparent layer.

In another preferred embodiment, in the step (6), the filter pack is capable of removing fat cells from the primary fat extract.

In another preferred embodiment, in the step (6), the filtration and sterilization are carried out through a filter (such as a 0.22 μm microporous filter membrane).

In another preferred embodiment, the filter is a microporous membrane filter.

In another preferred embodiment, the pore size of the microporous filter membrane is 0.05-0.8 μm, preferably 0.1-0.5 μm, more preferably 0.1-0.4 μm, more preferably 0.15-0.3 μm, more preferably 0.2-0.25 μm, and most preferably 0.22 μm.

In another preferred embodiment, in the step (6), the filtration and sterilization is carried out by first filtering through a first filter that can filter cells, and then through a second filter (such as a 0.22 μm filter) that can filter pathogens (such as bacteria).

In another preferred embodiment, the step (6) further includes subpackaging the fat extract to form a subpackaging product. The subpacked extract can be stored at −20° C. for later use; it can be used directly after thawing at low temperature (e.g. −4° C.) or at normal temperature, or stored at low temperature (e.g. 4° C.) for a period of time for later use after thawing.

In another preferred embodiment, the dosage form of the composition or preparation is a powder, a granule, a capsule, an injection, a tincture, an oral liquid, a tablet or a lozin.

In another preferred embodiment, the injection is an intravenous or intramuscular injection.

In another preferred embodiment, the dosage form of the composition or preparation is a solid dosage form, a semi-solid dosage form, or a liquid dosage form, such as a solution, gel, cream, emulsion, ointments, cream, paste, cake, powder, patch, etc.

A third aspect of the present invention provides a cell-free fat extract, the cell-free fat extract is obtained by the method of the second aspect of the present invention.

The fourth aspect of the present invention provides a composition or preparation, and the composition or preparation comprises (a) a cell-free fat extract of the third aspect of the present invention; and (b) a pharmaceutically, food, health care product or dietary acceptable carrier or excipient.

In another preferred embodiment, the composition is a pharmaceutical composition, a food composition, a health care composition or a dietary supplement.

In another preferred embodiment, the dosage form of the composition or preparation is a powder, a granule, a capsule, an injection, a tincture, an oral liquid, a tablet or a lozin.

In another preferred embodiment, the injection is an intravenous or intramuscular injection.

In another preferred embodiment, the dosage form of the composition or preparation is a solid dosage form, a semi-solid dosage form, or a liquid dosage form, such as a solution, gel, cream, emulsion, ointments, cream, paste, cake, powder, patch, etc.

In another preferred embodiment, the percentage by mass of the cell-free fat extract in the composition or preparation is 5 wt %, preferably 1-20 wt %, based on the total weight of the cosmetic composition.

The fifth aspect of the present invention provides a method of preparing a composition or preparation of the fourth aspect of the present invention, the method comprises the step of: mixing the cell-free fat extract of the third aspect of the present invention with a pharmaceutically, food, nutraceutical or dietary acceptable carrier or excipient to form the composition or preparation.

The sixth aspect of the present invention provides a method for preventing and/treating pulmonary fibrosis by administering the cell-free fat extract of the third aspect of the present invention to a subject in need thereof.

In another preferred embodiment, the subject is a human or non-human mammal.

In another preferred embodiment, the non-human mammal comprises a rodent, such as a rat, a mouse.

It should be understood that within the scope of the present invention, the above-mentioned technical features of the present invention and the technical features specifically described in the following (such as examples) can be combined with each other to form a new or preferred technical solution. Limited to space, it will not be repeated herein.

### DESCRIPTION OF THE DRAWINGS

FIG. 1 shows the effect of different groups on survival rate (%) of rats with pulmonary fibrosis in (mean values).

FIG. 2 shows the changes (x̅±SD) in body weight (g) over time of different groups of rats with pulmonary fibrosis.

FIG. 3 shows the lung weight index (%)(x̅±SD) of different groups of rats with pulmonary fibrosis.

FIG. 4 shows HE staining (200×) of the degree of inflammatory cell infiltration in the lung tissue of different groups of rats with pulmonary fibrosis.

FIG. 5 shows the Masson staining (200×) of the degree of fibrosis in the lung tissue of different groups of rats with pulmonary fibrosis.

FIG. 6 shows the results of histopathological evaluation of pulmonary tissue fibrosis in different groups of rats with pulmonary fibrosis.

### DETAILED DESCRIPTION OF EMBODIMENTS

After extensive and in-depth research, the present inventors have developed for the first time that the cell-free fat extract has excellent therapeutic effect on pulmonary fibrosis. On this basis, the present invention is completed.

Terms

Unless otherwise defined, all technical and scientific terms used herein have the same meaning as generally understood by those skilled in the art to which the present invention belongs.

As used herein, the terms “include”, “contain” and “comprise” are used interchangeably, including not only open definitions, but also semi-closed, and closed definitions. In other words, the term includes “consisting of” and “consisting essentially”.

As used herein, the terms “cell-free fat extract”, “cell free fat extract” and “CEFFE” are used interchangeably.

In the present invention, the term “prevention” means a method of preventing the onset of a disease and/or its accompanying symptoms or protecting a subject from developing the disease. The “prevention” used herein also includes delaying the onset of the disease and/or its accompanying symptoms and reducing the risk of disease in the subject.

The “treatment” described in the present invention includes delaying and terminating the progression of the disease, or eliminating the disease, and does not require 100% inhibition, elimination and reversal. In some embodiments, the composition or pharmaceutical composition of the present invention reduces, inhibits and/or reverses diabetes, for example, by at least about 10%, at least about 30%, at least about 50%, or at least about 80%, compared to the level observed in the absence of the cell-free fat extract, composition, kit, food or health care kit, active ingredient combination described herein.

As used herein, “improvement” includes prevention, treatment, mitigation, reversal and mitigation, etc.

As used herein, the term “IGF-1” is called insulin-like growth factors-1.

As used herein, the term “BDNF” is called brain-derived neurotrophic factor (BDNF).

As used herein, the term “GDNF” is called glial cell line-derived neurotrophic factor.

As used herein, the term “bFGF” is called basic fibroblast growth factor.

As used herein, the term “VEGF” is called vascular endothelial growth factor.

As used herein, the term “TGF-β1” is called transforming growth factor-β1.

As used herein, the term “HGF” is called hepatocyte growth factor.

As used herein, the term “PDGF” is called platelet-derived growth factor.

As used herein, the term “EGF” is called Epidermal Growth Factor.

As used herein, the term “NT-3” is called neurotrophins-3.

As used herein, the term “GH” is called Growth Hormone.

As used in the text, the term “G-CSF” is called granulocyte colony stimulating factor.

Cell Free Fat Extract (CEFFE) and Preparation Method Thereof

As used herein, the terms “cell-free fat extract of the present invention”, “extract of the present invention”, “fat extract of the present invention” and the like are used interchangeably to refer to an extract (or extract liquid) derived from fatty tissue prepared without adding any solutions, solvents, small molecules, chemicals, and biological additives during the preparation of the fat extract (other than the rinsing step). A typical process for preparing an extract of the present invention is as described above in the second aspect of the present invention. In addition, it should be understood that although the extract of the present invention does not need to add any additives (or additive ingredients) during the preparation process, some or a small amount of a safe substance (such as a small amount of water) that does not negatively or adversely affect the activity of the extract of the present invention can also be added.

The cell-free fat extract described in the present invention may include a variety of cytokines. Typically, the cell-free fat extract comprises one or more of IGF-1, BDNF, GDNF, TGF-β, HGF, bFGF, VEGF, TGF-β1, HGF, PDGF, EGF, NT-3, GH, and G-CSF.

Preferably, the cell-free fat extract of the present invention is obtained by the method as described above in the second aspect of the present invention.

Typically, the cell-free fat extract described in the present invention is prepared by the following methods:

(1) providing an fatty tissue raw material, crushing the fatty tissue raw material and rinsing it (e.g., with normal saline) to obtain a rinsed fatty tissue;

(2) centrifuging the rinsed fatty tissue to obtain a layered mixture;

(3) for the layered mixture, the upper oil layer and the lower water layer are removed, and collecting the intermediate layer (i.e. the fat layer containing fat cells);

(4) emulsifying the intermediate layer to obtain an emulsified fat mixture (also called nano-fat);

(5) centrifuging the emulsified fat mixture, thereby obtaining an intermediate liquid layer, i.e. a primary fat extract; and

(6) filtering and de-bacterizing the primary fat extract, thereby obtaining the cell-free fat extract.

Specifically, the cell-free fat extract of the present invention

In another preferred embodiment, in the step (2), the centrifugation is performed at 800-2500 g, preferably 800-2000 g, more preferably 1000-1500 g, and most preferably 1100-1300 g.

In another preferred embodiment, in the step (2), the centrifugation time is 1-15 min, preferably 1-10 min, more preferably 1-8 min, and most preferably 1-5 min.

In another preferred embodiment, in the step (4), the emulsification is mechanical emulsification.

In another preferred embodiment, the mechanical emulsification is performed by repeated blowing by a syringe (e.g., blowing 20-200 times, preferably 20-150 times, more preferably 20-100 times, more preferably 30-50 times).

In another preferred embodiment, the blowing method is that two 10 ml injection syringes are connected to a tee tube and repeatedly push at a constant speed.

In another preferred embodiment, in the step (4), the emulsification is by means of crushing through a tissue homogenizer.

In another preferred embodiment, the step (5) further includes freezing and thawing the emulsified fat mixture before the centrifugation treatment.

In another preferred embodiment, the thawed mixture is used for centrifugation after freezing and thawing treatment.

In another preferred embodiment, the freezing temperature is from −50° C. to −120° C., preferably from −60° C. to −100° C., more preferably from −70° C. to −90° C.

In another preferred embodiment, the thawing temperature is 20-40° C., preferably 25-40° C., more preferably 37° C.

In another preferred embodiment, the number of cycles of thawing after freezing is 1-5 (preferably 1, 2, 3 or 4).

In another preferred embodiment, in the step (5), after centrifugation, the emulsified fat mixture is layered into four layers, the first layer is an oil layer, the second layer is a residual fatty tissue layer, the third layer is a liquid layer (i.e., an intermediate liquid layer), and the fourth layer is a cell/tissue debris precipitation layer.

In another preferred embodiment, in the step (5), the centrifugation is performed at 800-2500 g, preferably 800-2000 g, more preferably 1000-1500 g, and most preferably 1100-1300 g.

In another preferred embodiment, in the step (5), the centrifugation time is 1-15 min, preferably 1-10 min, more preferably 2-8 min, and most preferably 3-7 min.

In another preferred embodiment, in the step (5), the first layer, the second layer, the third layer and the fourth layer are sequentially arranged from top to bottom.

In another preferred embodiment, in the step (5), the intermediate liquid layer is a transparent or substantially transparent layer.

In another preferred embodiment, in the step (6), the filter pack is capable of removing fat cells from the primary fat extract.

In another preferred embodiment, in the step (6), the filtration and sterilization are carried out through a filter (such as a 0.22 μm microporous filter membrane).

In another preferred embodiment, the filter is a microporous membrane filter.

In another preferred embodiment, the pore size of the microporous filter membrane is 0.05-0.8 μm, preferably 0.1-0.5 μm, more preferably 0.1-0.4 μm, more preferably 0.15-0.3 μm, more preferably 0.2-0.25 μm, and most preferably 0.22 μm.

In another preferred embodiment, in the step (6), the filtration and sterilization is carried out by first filtering through a first filter that can filter cells, and then through a second filter(such as a 0.22 μm filter) that can filter pathogens (such as bacteria).

In another preferred embodiment, the step (6) further includes subpackaging the fat extract to form a subpackaging product. The subpacked extract can be stored at −20° C. for later use; it can be used directly after thawing at low temperature (e.g. −4° C.) or at normal temperature, or stored at low temperature (e.g. 4° C.) for a period of time for later use after thawing.

Pulmonary Fibrosis

Pulmonary fibrosis (PF) is a disease characterized by diffuse pneumonia and structural disorders of the alveoli that culminate in interstitial fibrosis, a serious pathological feature common to a group of clinical conditions known as interstitial lung disease. Interstitial lung disease can be classified into seven categories: primary lung, accompanied by systemic rheumatic disease, drug- or radiation-induced disease, accompanied by environmental or occupational disease, accompanied by pulmonary vascular disease, alveolar stasis disease, and genetic disease. According to the causes, they can be divided into two categories: idiopathic and secondary. The common feature is that the normal alveolar structure is damaged by inflammation due to various causes, i.e., pulmonary alveolitis; the damage is repaired by the accumulation of collagen scar tissue, i.e., fibrosis, and the lung tissue gradually loses its normal respiratory function, resulting in dyspnea, hypoxia, and other symptoms, which eventually leads to respiratory failure. The incidence of pulmonary fibrosis, especially idiopathic pulmonary fibrosis, has been increasing in recent years due to various causes.

Pulmonary fibrosis can be caused by a variety of factors, for example, by cytotoxic antibiotics, chemotherapeutic drugs, or other drugs.

Typically, the pulmonary fibrosis is caused by cytotoxic antibiotics (e.g., bleomycin).

Typically, the pulmonary fibrosis is caused by anti-tumor drugs (such as the chemotherapeutic drug bleomycin).

Typically, the pulmonary fibrosis is bleomycin-induced or -caused pulmonary fibrosis.

Use

The cell-free fat extract of the present invention is effective in the prevention and/treatment of pulmonary fibrosis.

Typically, the prevention and/or treatment of pulmonary fibrosis comprises prevention and/or treatment by one or more ways selected from the group consisting of:

(i) reducing lung weight index;

(ii) improving peribronchial, bronchio-interstitial and/or pulmonary alveolar inflammatory cell infiltration of a lung;

(iii) improving peribronchial and/or bronchio-interstitial fibrosis of a lung; and/or

(iv) improving peribronchial edema of a lung.

The present invention also provides a method of preventing and/or treating pulmonary fibrosis, comprising the step of administering to a subject in need thereof a cell-free fat extract of the present invention.

In another preferred embodiment, the subject is a human or non-human mammal.

In another preferred embodiment, the non-human mammal comprises a rodent, such as a rat, a mouse.

Composition and Administration

The compositions described herein include, but are not limited to, pharmaceutical compositions, food compositions, health-care compositions, dietary supplements, and the like.

Typically, the cell-free fat extract of the present invention can be prepared as pharmaceutical compositions in dosage forms such as tablets, capsules, powders, microgranule, solutions, lozenges, jellies, cream, spiritus, suspensions, tinctures, mud dressings, liniment, lotions, and aerosols, etc. Pharmaceutical compositions can be prepared by commonly known preparation techniques, and suitable pharmaceutical additives can be added to the drug.

The compositions of the present invention can also include pharmaceutically, food, nutraceutical or dietary acceptable carriers. “Pharmaceutically, food, nutraceutical or dietary acceptable carriers” means one or more compatible solid or liquid filler or gel substances that are suitable for human use and must have sufficient purity and sufficiently low toxicity. “Compatibility” herein refers to the ability of components of a composition to blend with the compounds of the invention and with each without significantly reducing the efficacy of the compounds. Examples of pharmaceutically, food, nutraceutical or dietary acceptable carriers include cellulose and its derivatives (such as sodium carboxymethyl cellulose, sodium ethyl cellulose, cellulose acetate, etc.), gelatin, talc, solid lubricants (such as stearic acid, Magnesium stearate), calcium sulfate, vegetable oil (such as soybean oil, sesame oil, peanut oil, olive oil, etc.), polyols (such as propylene glycol, glycerin, mannitol, sorbitol, etc.), emulsifier (such as Tween®), wetting agents (such as sodium dodecyl sulfate), colorants, flavoring agents, stabilizers, antioxidants, preservatives, pyrogen-free water, etc.

The methods of administration of the compositions of the present invention are not particularly limited, and representative methods of administration include, but are not limited to, oral, parenteral (intravenous, intramuscular), topical administration, preferably oral administration and injection administration.

The dosage form of the composition or preparation described in the present invention is an oral preparation, a topical preparation or an injection preparation. Typically, solid dosage forms for oral administration include capsules, tablets, pills, powders and granules. In these solid dosage forms, the active compounds is mixed with at least one conventional inert excipient (or carrier), such as sodium citrate or dicalcium phosphate, or mixed with: (a) fillers or compatibilizers, e.g., starch, lactose, sucrose, glucose, mannitol and silicic acid; (b) binders, e.g., hydroxymethylcellulose, alginate, gelatin, polyvinylpyrrolidone, sucrose, and gum arabic; (c) humectants, e.g., glycerol; (d) disintegrants, e.g., agar, calcium carbonate, potato starch or tapioca starch, alginic acid, certain complex silicates, and sodium carbonate;(e) dissolution-retarding agents, e.g., paraffin; (f) absorption accelerators, e.g., quaternary amine compounds; (g) wetting agents, e.g., cetearyl alcohol and glycerol monostearate; (h) sorbents, e.g., kaolin; and (i) lubricants, e.g., talc, calcium stearate, magnesium stearate, solid polyethylene glycol, sodium dodecyl sulfate, or mixtures thereof. In capsules, tablets and pills, dosage forms may also contain buffers.

Solid dosage forms such as tablets, sugar pills, capsules and granules may be prepared using coating and shell materials such as casing and other materials well known in the art. They can contain opaque agents.

Liquid dosage forms for oral administration include pharmaceutically acceptable emulsions, solutions, suspensions, syrups or tinctures. In addition to the active compounds, the liquid dosage form may contain inert diluents conventionally used in the art, such as water or other solvents, solubilizers and emulsifiers, for example, ethanol, isopropanol, ethyl carbonate, ethyl acetate, propylene glycol, 1,3-butanediol, dimethylformamide and oils, especially cottonseed oil, peanut oil, corn germ oil, olive oil, castor oil and sesame oil, or mixtures thereof.

In addition to these inert diluents, the composition may also contain auxiliaries such as wetting agents, emulsifiers, suspending agents, sweeteners, flavoring agents and flavors.

In addition to the active ingredient, the suspension may comprise suspending agents, such as ethoxylated isooctadecanol, polyoxyethylene sorbitol and dehydrated sorbitol esters, microcrystalline cellulose, methanolic aluminum, agar, and any mixtures thereof.

The composition for parenteral injection may comprise physiologically acceptable sterile aqueous or anhydrous solutions, dispersions, suspensions or emulsions, and sterile powders for redissolution into sterile injectable solutions or dispersions. Suitable aqueous and non-aqueous carriers, diluents, solvents, or excipients include water, ethanol, polyols, and suitable mixtures thereof.

Dosage forms of the compounds of the invention for topical administration include ointments, powder, patches, sprays and inhalants. The active ingredient is mixed under sterile conditions with a physiologically acceptable carrier and any preservatives, buffers or propellants as may be required.

The cell-free fat extract of the present invention can be administered alone, or in combination with other drugs for the prevention and/or treatment of fatty liver and/or its complications.

When the composition is administered, a safe and effective amount of the cell-free fat extract of the present invention is applied to a human or non-human animal in need of treatment (e.g., rat, mouse, dog, cat, cow, chicken, duck, etc.) at a dose that is pharmaceutically, food or dietary acceptable to the effective administration. As used herein, the term “safe and effective amount” refers to an amount that produces function or activity to humans and/or animals and is acceptable to humans and/or animals. Those ordinary skilled in the art will understand that the “safe and effective amount” described may vary depending on the form of the pharmaceutical composition, the route of administration, the excipient of the drug used, the severity of the disease, and the combination with other drugs. For example, for a person of 60 kg body weight, the daily dose is usually 0.1 to 1000 mg, preferably 1 to 600 mg, more preferably 2 to 300 mg. Of course, the specific dosage should also consider the route of administration, the patient's health and other factors, which are within the skill range of skilled doctors.

The main advantages of the present invention include:

The present invention is the first to discover that cell-free fat extracts have excellent therapeutic effects on pulmonary fibrosis.

The present invention is further described below in conjunction with specific examples. It is to be understood that these examples are intended to illustrate the invention only and not to limit the scope of the invention. The following examples do not indicate the specific conditions of the experimental method, usually according to the conventional conditions, or according to the conditions recommended by the manufacturer. Unless otherwise specified, percentages and parts are calculated by weight.

### Example 1

1 Experimental Method

1.1. Preparation of Cell Free Fat Extract (CEFFE)

Fat is obtained by volunteers with informed consent. The preparation method of cell free fat tissue extract is as follows:

(1) Fatty tissue was obtained from 6 healthy women who underwent conventional liposuction, with an average age of 31 years (24-36 years). After anesthesia with local injection of swelling solution, a 3 mm liposuction aspiration cannula with a large lateral hole (2 mm×7 mm) connected to a 20 mL syringe was used, and the obtained fat was manually aspirated radially under negative pressure, and the fat was left upright and stationary, and after removal of the swelling solution, it was rinsed 3 times with saline.

(2) The rinsed fatty tissue was taken, placed in a centrifuge tube, then placed in a centrifuge, and centrifuged at 1200 g 4° C. for 3 minutes to obtain a layered mixture.

(3) For the layered mixture, the upper oil layer and the lower water layer were removed and the intermediate layer (i.e. the fat layer containing fat cells) was collected.

(4) For the intermediate layer, two 10 ml syringes connected to a tee tube were pushed repeatedly and uniformly for 30 times, thus performing mechanical emulsification and obtaining a mechanically emulsified fat mixture (also called nano-fat).

(5) The mechanically emulsified fat mixture was placed into a −80° C. refrigerator for freezing, and then thawed in a 37° C. water bath, and after a single freeze-thaw cycle, the thawed fat mixture was centrifuged at 1200 g 4° C. for 5 minutes to obtain a layered mixture, which was divided into 4 layers, the first layer being the oil layer, the second layer being the residual fatty tissue layer, the third layer being the liquid layer, and the fourth layer being the cell/tissue debris precipitation layer, the oil layer and the residual fatty tissue layer were removed and the liquid layer was aspirated, avoiding contamination of the cellular/tissue debris precipitation layer during the aspiration process, resulting in a primary fat extract.

(6) The obtained primary fat extract was filtered and de-bacterized through a 0.22 μm filter, thereby sterilizing and removing any live cells that may have been mixed, resulting in the cell-free fat extract (CEFFE) that was subpackaged and stored frozen at −20° C. and thawed at 4° C. when used.

For the prepared cell-free fat extract, the content of growth factors, including IGF-1, BDNF, GDNF, bFGF, VEGF, TGF-β, HGF, PDGF and other cytokines, was detected by ELISA immunosorbent assay kit. The average concentrations of 6 samples were as follows: IGF-1 (9840.6 pg/ml), BDNF (1764.5 pg/ml), GDNF (1831.9 pg/ml), bFGF (242.3 pg/ml), VEGF (202.9 pg/ml), TGF-β1 (954.5 pg/ml), HGF (898.4 pg/ml), PDGF (179.9 pg/ml).

1.2 Establishment and Grouping of Pulmonary Fibrosis Model in Rats

Thirty male SD rats were randomly divided into model control group (9 rats), CEFFE low-dose group (7 rats), CEFFE medium-dose group (7 rats) and CEFFE high-dose group (7 rats) according to their body weight measured before grouping, and all 30 animals were given the modeling reagent (bleomycin) by intra-airway nebulization. All animals were given bleomycin (7 mg/kg,1 mL/kg) by intra-airway nebulization on Day 1 for model establishment.

Day 1: animals were anesthetized by isoflurane inhalation, then fixed on a rat fixator placed at 45°, using a small animal anesthesia laryngoscope, pressing the root of the animal's tongue, exposing the glottis, gently inserting the needle (blunt) of the pulmonary miniature liquid nebulizer that drawn a quantitative amount of bleomycin solution into the trachea about 1 cm, then quickly pushing the plunger to nebulize the bleomycin solution into the lungs, quickly pulling out the needle, removing the animal from the fixator with the head facing upward and rotating it from side to side so that bleomycin was distributed as evenly as possible throughout the lung lobes. The dosing volume was kept to integer digits, and the dosing volume was aspirated at the upper index value when the dosing volume was between the two index volume lines.

After all animal models were established, the test product or solvent control was administered according to Table 1 below.

TABLE 1

| Group | | Administration | |
|---|---|---|---|
| number | Group | volume (mL/kg) | Dose (mg/kg) |
| 1 | Normal saline control group | 5 | 0 |
| 2 | CEFFE low-dose group | 2.5 | 9.28 |
| 3 | CEFFE medium-dose group | 5 | 18.55 |
| 4 | CEFFE high-dose group | 15 | 59.25 |

Day 1 to Day 28: routine animal feeding and general clinical observation were recorded.

Route of administration: intravenous injection

Frequency and duration of administration: Day 2, Day 6, Day 10, Day 14, Day 18, Day 22, Day 26, once every 4 days for a total of 7 times.

Intravenous administration: according to the recently weighed animal weight, the dosage of each animal was aspirated by disposable sterile syringe and administered by slow (about 10˜80 seconds) injection through the tail vein. The dosing volume was retained to 1 decimal place, and the dosing volume was aspirated according to the upper indexed value when the dosing volume was between the two indexed volume lines.

Animals were weighed on the day of grouping, on the day of modeling, on the day of administration, and before euthanasia to evaluate weight changes.

1.3 Lung Weight Index and Histopathological Tests

Animals were weighed on the day of grouping, on the day of modeling, on the day of administration, and before euthanasia. All animals were dissected and tissue was preserved on Day28. Animals were observed for abnormalities in the lungs, trachea, and bronchi. The lungs were separated and tissues such as fat were removed, the surface fluid was dried by filter paper, and the lungs were weighed and used to calculate the lung weight index, lung weight index (%)=lung weight/body weight×100%.

The right lung and bronchioles were fixed in 10% neutral buffer formalin solution, paraffin embedded, sectioned, filmed, and HE stained for evaluation of inflammatory cell infiltration degree in lung tissue, and Masson staining for evaluation of fibrosis degree in lung tissue. The levels of histopathological evaluation were as follows:

Grade 1: slight=lesions are barely visible and/or few/involved areas or lesions areas are very small.

Grade 2: mild=lesions are readily visible but only apparent due to few lesions/involved areas or lesions areas are small.

Grade 3: moderate=lesions are clearly present and evaluable in terms of size and/or number.

Grade 4: severe=large lesion area or large lesion size.

1.4 Statistical Analysis

Data acquisition: data were collected by system generation and manual recording.

Data analysis: the statistical software SPSS 13.0 or GraphPad Prism 5 used in this experiment processed the data. All statistical analyses were performed using two-tailed analysis with a statistical level of P≤0.05. The data were expressed as “mean±standard deviation”.

2. Results

2.1 CEFFE Treatment Improves Survival Rate and Weight of Rats with Pulmonary Fibrosis Model

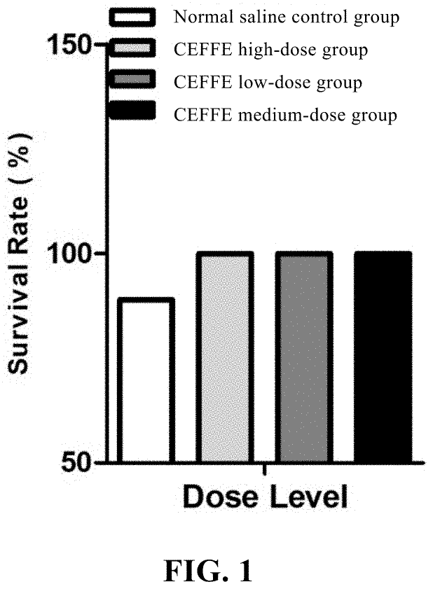

The survival rates of different groups of rats with pulmonary fibrosis model at Day28 are shown in FIG. 1, from which it can be seen that the survival rates of animals in saline control group, CEEFE low-dose group, CEEFE medium-dose group and CEEFE high-dose group were 89%, 100%, 100% and 100%, respectively, and the survival rates of animals in each CEEFE group were higher than those in the model control group, indicating that CEEFE can significantly improve the survival rate of rats with pulmonary fibrosis model.

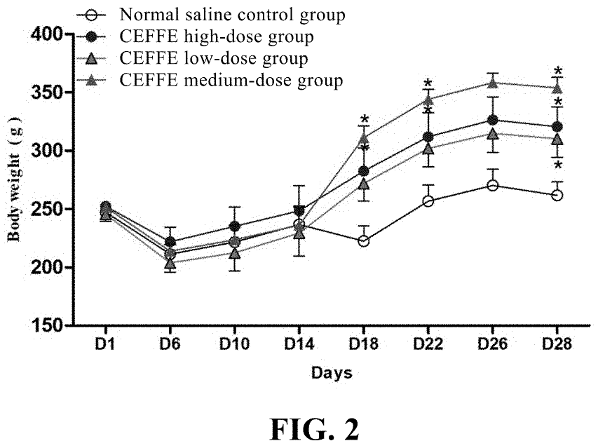

The changes of body weight over time of different groups of rats with pulmonary fibrosis model are shown in FIG. 2, from which it can be seen that the average body weight of animals in all groups decreased on Day 6 after modeling, after which the average body weight of all groups increased steadily with the extension of the test period; compared with animals in the model control group, the average body weight of animals in the CEFFE low-dose, CEFFE medium-dose and CEFFE high-dose groups were more significantly increased on Day 18, Day 22, Day 26 and Day 28 with statistical differences, and the results in FIG. 2 indicated that CEEFE could significantly improve the body weight of rats with pulmonary fibrosis model.

2.2 CEFFE Treatment Reduces Lung Weight Index of Rats with Pulmonary Fibrosis Model

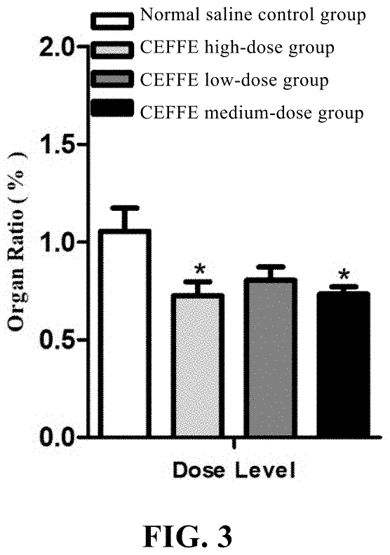

The lung weight index (%) of rats with pulmonary fibrosis in different groups is shown in FIG. 3, from which it can be seen that the lung weight index of animals in the CEFFE low-dose group, CEFFE medium-dose and CEFFE high-dose groups were reduced compared with the control group, which was statistically different.

2.3 CEFFE Treatment Improves Pulmonary Inflammation in Model Rats

During this test, the pulmonary fibrosis caused by intra-airway nebulized bleomycin was mainly visible microscopically as peribronchial/interstitial/alveolar infiltration of inflammatory cells dominated by single nucleated cells, neutrophils, macrophages, peribronchial/interstitial fibrosis and peribronchial edema in the lungs. Alveolar hemorrhage, pulmonary artery embolism and alveolar dilatation were partially seen, with peribronchial/interstitial/alveolar inflammatory cell infiltration, peribronchial/interstitial fibrosis and peribronchial edema dominated by single nucleated cells, neutrophils, macrophagesbeing the commonly seen lesions in the model control group.

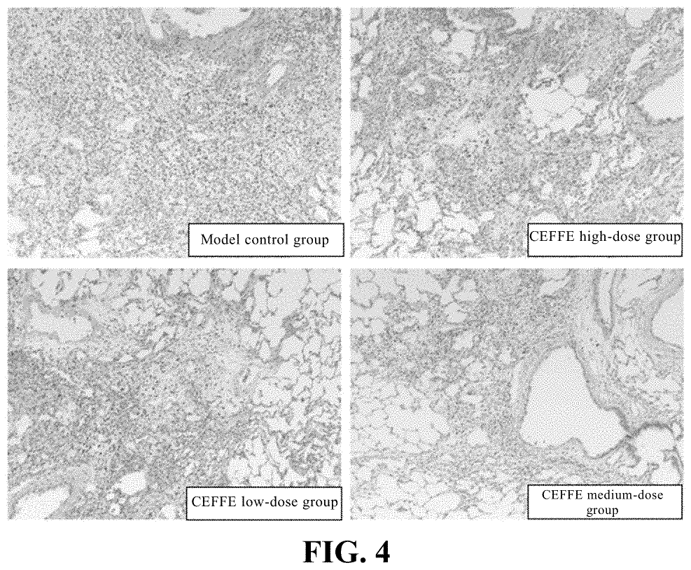

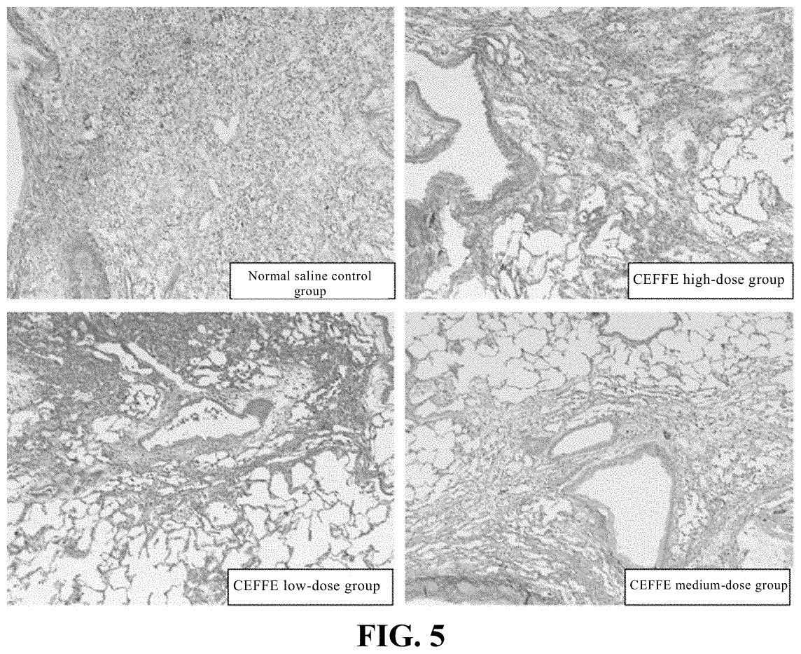

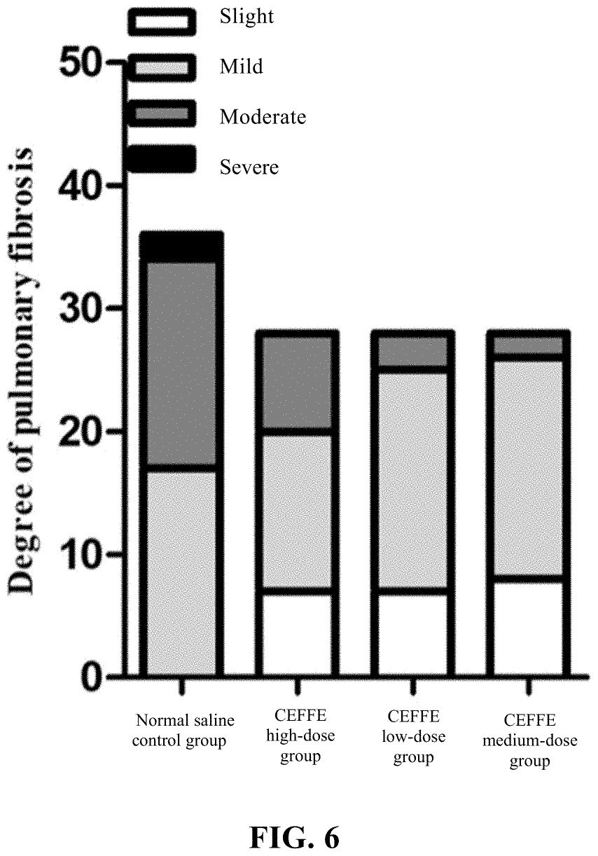

HE staining of the degree of inflammatory cell infiltration in lung tissue of different groups of rats with pulmonary fibrosis are shown in FIG. 4, Masson staining of the degree of fibrosis in lung tissue of different groups of rats with pulmonary fibrosis are shown in FIG. 5, and the results of histopathological evaluation of fibrosis in lung tissue of different groups of rats with pulmonary fibrosis are shown in FIG. 6. Compared with the normal saline control group, each CEFFE treatment group showed significant improvement in pulmonary peribronchial/interstitial/alveolar inflammatory cell infiltration, peribronchial/interstitial fibrosis and peribronchial edema, and there was a certain dose relationship between the low and medium dose groups of CEFFE and all of them were better than the improvement effect of the high dose group.

### CONCLUSION

The example used intra-airway nebulized administration of bleomycin to establish pulmonary fibrosis rat model, and treated with intravenous CEFFE, and confirmed that CEFFE could effectively improve the survival rate and body weight of the model rats, reduce the lung weight index, and reduce the inflammation and damage of alveolar tissues, suggesting that CEFFE treatment could effectively improve the pulmonary fibrosis and systemic status of the model rats, indicating that CEFFE has excellent therapeutic effects on pulmonary fibrosis.

All documents referred to in the present invention are incorporated by reference herein as if each document is individually incorporated by reference. Further, it should be understood that upon reading the above teaching of the present invention, various variations or modifications may be made to the present invention by those skilled in the art, and those equivalents also fall within the scope defined by the appended claims of the present application.

1. A Use of a cell-free fat extract in the preparation of a composition or preparation for prevention and/or treatment of pulmonary fibrosis.

2. The use of claim 1, wherein the pulmonary fibrosis comprises pulmonary fibrosis caused by a cytotoxic antibiotic.

3. The use of claim 2, wherein the cytotoxic antibiotic comprises bleomycin.

4. The use of claim 1, wherein the pulmonary fibrosis comprises pulmonary fibrosis caused by an anti-tumor drug.

5. The use of claim 1, wherein the prevention and/or treatment of pulmonary fibrosis comprises one or more ways of prevention and/or treatment selected from the group consisting of:
(i) reducing lung weight index;
(ii) improving peribronchial, bronchio-interstitial and/or pulmonary alveolar inflammatory cell infiltration of a lung;
(iii) improving peribronchial and/or bronchio-interstitial fibrosis of a lung; and/or
(iv) improving peribronchial edema of a lung.

6. The use of claim 1, wherein the cell-free fat extract comprises one or more components selected from the group consisting of IGF-1, BDNF, GDNF, HGF, bFGF, VEGF, TGF-β1, HGF, PDGF, EGF, NT-3, GH, G-CSF, and combinations thereof.

7. The use of claim 6, wherein the cell-free fat extract comprises one or more features selected from the group consisting of:
in the cell-free fat extract, the concentration of the IGF-1 is 5000-30000 pg/ml, preferably 6000-20000 pg/ml, more preferably 7000-15000 pg/ml, more preferably 8000-12000 pg/ml, more preferably 9000-11000 pg/ml, more preferably 9500-10500 pg/ml;
in the cell-free fat extract, the concentration of BDNF is 800-5000 pg/ml, preferably 1000-4000 pg/ml, more preferably 1200-2500 pg/ml, more preferably 1400-2000 pg/ml, more preferably 1600-2000 pg/ml, more preferably 1700-1850 pg/ml;
in the cell-free fat extract, the concentration of GDNF is 800-5000 pg/ml, preferably 1000-4000 pg/ml, more preferably 1200-2500 pg/ml, more preferably 1400-2000 pg/ml, more preferably 1600-2000 pg/ml, more preferably 1700-1900 pg/ml;
in the cell-free fat extract, the concentration of bFGF is 50-600 pg/ml, preferably 100-500 pg/ml, more preferably 120-400 pg/ml, more preferably 150-300 pg/ml, more preferably 200-280 pg/ml, more preferably 220-260 pg/ml;
in the cell-free fat extract, the concentration of VEGF is 50-500 pg/ml, preferably 100-400 pg/ml, more preferably 120-300 pg/ml, more preferably 150-250 pg/ml, more preferably 170-230 pg/ml, more preferably 190-210 pg/ml;
in the cell-free fat extract, the concentration of TGF-β1 is 200-3000 pg/ml, preferably 400-2000 pg/ml, more preferably 600-1500 pg/ml, more preferably 800-1200 pg/ml, more preferably 800-1100 pg/ml, more preferably 900-1000 pg/ml;
in the cell-free fat extract, the concentration of HGF is 200-3000 pg/ml, preferably 400-2000 pg/ml, more preferably 600-1500 pg/ml, more preferably 600-1200 pg/ml, more preferably 800-1000 pg/ml, more preferably 850-950 pg/ml; and/or
in the cell-free fat extract, the concentration of PDGF is 50-600 pg/ml, preferably 80-400 pg/ml, more preferably 100-300 pg/ml, more preferably 140-220 pg/ml, more preferably 160-200 pg/ml, more preferably 170-190 pg/ml.

8. The use of claim 6, wherein the cell-free fat extract comprises one or more features selected from the group consisting of:
the weight ratio of the IGF-1 to VEGF is 20-100:1, preferably 30-70:1, more preferably 40-60:1, and most preferably 45-55:1;
the weight ratio of BDNF to VEGF is 2-20:1, preferably 4-15:1, more preferably 6-12:1, and most preferably 8-9.5:1;
the weight ratio of GDNF to VEGF is 2-20:1, preferably 4-15:1, more preferably 6-12:1, and most preferably 8.5-9.5:1;
the weight ratio of bFGF to VEGF is 0.2-8:1, preferably 0.5-5:1, more preferably 0.6-2:1, more preferably 0.8-1.6:1, and most preferably 1-1.5:1;
the weight ratio of TGF-β1 to VEGF is 1-20:1, preferably 1-15:1, more preferably 1-10:1, more preferably 2-8:1, more preferably 4-6:1;
the weight ratio of HGF to VEGF is 1-20:1, preferably 1-15:1, more preferably 1-10:1, more preferably 2-8:1, more preferably 4-5.5:1; and/or
the weight ratio of PDGF to VEGF is 0.1-3:1, preferably 0.2-2:1, more preferably 0.4-1.5:1, and most preferably 0.7-1.2:1.

9. The use of claim 1, wherein the cell-free fat extract is prepared by the following method:
(1) providing an fatty tissue raw material, crushing the fatty tissue raw material and rinsing it (e.g., with normal saline) to obtain a rinsed fatty tissue;
(2) centrifuging the rinsed fatty tissue to obtain a layered mixture;
(3) for the layered mixture, the upper oil layer and the lower water layer are removed, and collecting the intermediate layer (i.e. the fat layer containing fat cells);
(4) emulsifying the intermediate layer to obtain an emulsified fat mixture (also called nano-fat);
(5) centrifuging the emulsified fat mixture, thereby obtaining an intermediate liquid layer, i.e. a primary fat extract; and
(6) filtering and de-bacterizing the primary fat extract, thereby obtaining the cell-free fat extract.

10. The use according to claim 1, wherein the composition or preparation is a pharmaceutical composition or preparation, a food composition or preparation, a health care product composition or preparation, or a dietary supplement.

11. A cell-free fat extract, wherein the cell-free fat extract is prepared by the following method:
(1) providing an fatty tissue raw material, crushing the fatty tissue raw material and rinsing it (e.g., with normal saline) to obtain a rinsed fatty tissue;
(2) centrifuging the rinsed fatty tissue to obtain a layered mixture;
(3) for the layered mixture, the upper oil layer and the lower water layer are removed, and collecting the intermediate layer (i.e. the fat layer containing fat cells);
(4) emulsifying the intermediate layer to obtain an emulsified fat mixture (also called nano-fat);
(5) centrifuging the emulsified fat mixture, thereby obtaining an intermediate liquid layer, i.e. a primary fat extract; and
(6) filtering and de-bacterizing the primary fat extract, thereby obtaining a cell-free fat extract.

12. A method of preventing and/or treating pulmonary fibrosis, comprising the step of administering the cell-free fat extract of claim 11 to a subject in need thereof.
